(19) United States
(12) Patent Application Publication
(10) Pub. No.: US 2023/0408838 A1
(43) Pub. Date: Dec. 21, 2023

(54) PERFUSION SYSTEMS FOR DRIFT-FREE MICROSCOPY

(71) Applicant: FEI DEUTSCHLAND GMBH, Dreieich (DE)

(72) Inventors: Rainer DAUM, Gräfelfing (DE); Meike PEDERSEN, Gräfelfing (DE)

(21) Appl. No.: 18/032,540

(22) PCT Filed: Oct. 19, 2021
(86) PCT No.: PCT/IB2021/059632

§ 371 Date: Apr. 18, 2023

Related U.S. Application Data
(60) Provisional application No. 63/104,418, filed on Oct. 22, 2020.

(51) Int. Cl. G02B 27/58; G02B 21/34; C12M 1/00

(52) U.S. Cl. CPC G02B 27/58; G02B 21/34; C12M 29/10

(57) ABSTRACT

A perfusion system for reducing or eliminating sample drift during microscopy imaging includes a sample chamber that has an inlet opening associated therewith. The perfusion system also includes a reservoir, which is in fluid communication with the inlet opening and positioned above the inlet opening in a direction opposite the force of gravity. The perfusion system includes an outlet opening associated with the sample chamber. Furthermore, the perfusion system includes a wick. A first portion of the wick forms a fluid-tight connection with the outlet opening. A second portion of the wick is disposed within a waste tank. A capillary tension of the wick contributes to a laminar flow of fluid across an optical detection area of the sample chamber.

### CROSS-REFERENCE TO RELATED APPLICATION

This application is a national stage application, filed under 35 U.S.C § 371, of International Patent Application PCT/IB2021/059632, filed on Oct. 19, 2021, which claims the benefit of priority to U.S. Provisional Application No. 63/104,418, filed on Oct. 22, 2020, which is incorporated by reference herein in its entirety.

### BACKGROUND

### Technical Field

The present application relates generally to microscopy and imaging systems used therein. More particularly, the present application relates to apparatuses, systems, and components associated with perfusion systems for facilitating drift-free microscopy.

### INTRODUCTION

Microscopy is concerned with observing small, often microscopic, objects, such as live cells. Some imaging systems used in microscopy include perfusion systems that are operable to perfuse imaged samples with liquid media. Perfusing samples, such as live cells, with liquid media may be performed in association with live cell microscopy for a variety of purposes, such as providing live cells with a constant supply of fresh growth media and thereby prevent the formation of any imaging artifacts resulting from a lack of proper nutrients or exposure to metabolic byproducts accumulating in stagnant media. Perfusion can also be useful to disperse fluorescent probes over live cells (e.g., in preparation for fluorescent imaging) or to wash or remove media from cells without leaving an optical detection area. Perfusing live cells with liquid media (e.g., that includes a buffer, stimulus, and/or insult) may be desirable while performing live cell imaging as it allows researchers to immediately view and capture images of a sample in response to perfused media. For such and other purposes, perfusion systems that provide an even flow through an associated sample chamber are advantageous. For example, an even flow of media through the sample chamber of a perfusion system may enable even distribution of a growth medium, buffer, fluorescent probe, stimulus, and/or insult over a sample.

However, conventional perfusion systems and techniques suffer from a number of shortcomings. For example, some conventional perfusion systems use electrically-driven pumps to perfuse media through the sample chamber. However, the operation of electrically-driven pumps can cause mechanical vibrations that interfere with image capture events in optical high-resolution or super-resolution microscopy. In addition, pump effects and/or cycles may cause hydrodynamic fluctuations and flow variations within the sample chamber (e.g., at short time scales), which can negatively affect the accuracy, resolution, or feasibility of imaging studies, particularly time lapse imaging studies. Furthermore, electrical perfusion pumps may create pressure within the sample chamber that causes deformation or loss of cellular adherence and/or causes the sample to drift within the viewing area. Still furthermore, electrically-driven perfusion pumps may dissipate heat into the perfusion media and consequently the sample chamber, which may affect the thermal characteristics of the sample and/or may influence the biochemical reactivity exhibited by the sample. In this regard, perfusion systems operated via electrically-driven pumps are often unsuitable for high-resolution microscopy and/or long-term imaging experiments.

Other conventional systems and techniques for perfusing a sample in a microscopy context utilize a syringe and/or pipette to inject media through a sample chamber inlet and into the sample chamber. However, mechanical position changes of a pipette or syringe create pressure differentials at the sample inlet and outlet, thereby causing drift and/or deformation of the sample chamber, which can affect the optical properties of the sample chamber. Furthermore, such perfusion systems and techniques may result in a nozzle effect that leads to turbulent flow throughout the sample chamber, leading to an uneven distribution of perfused media within the sample chamber. In addition, syringe- and/or pipette-driven perfusion systems and techniques preclude sealing the sample chamber against ambient gases. For example, some experiments rely on limiting oxygen exposure to the sample or perfusion media (e.g., to induce hypoxia, prevent photobleaching of certain oxygen-reactive fluorescence dyes, etc.), and the open chamber/inlet port can make it difficult to control exposure of the sample chamber/perfusion liquid to ambient gases.

Conventional perfusion systems and techniques for microscopy applications are also typically unsuitable for implementation with 3D localization microscopy. For example, confocal microscopy is an optical imaging technique that uses laser scanning at different depths to build a high-resolution and high-contrast 3D reconstruction of a sample. Confocal microscopy (and other scanning techniques) may be implemented in standard microscope systems (configured for imaging a sample from one side thereof) or 4-pi microscope systems (configured for imaging a sample from opposing sides thereof).

3D localization microscopy relies on low drift (or drift-free) imaging conditions and an even spatial pressure gradient within the sample volume to collect optical high-resolution or super-resolution images of a sample. However, as described above, conventional perfusion systems and techniques are associated with sample drift, uneven dynamic pressure gradients, and/or vibrations within the sample volume, rendering such perfusion systems and techniques unsuitable for collecting sample images over long acquisition times, especially for 3D localization microscopy. Furthermore, at least some conventional perfusion systems and techniques rely on access to the sample from one side thereof near the region of interest, precluding implementation of these systems within 4-pi microscopy workflows.

Accordingly, there are a number of problems and disadvantages with existing perfusion systems that can be addressed.

### BRIEF SUMMARY

Various embodiments disclosed herein are related to apparatuses, systems, and components associated with perfusion systems for facilitating drift-free microscopy. Such embodiments may beneficially improve microscopy systems, such as 3D localization microscopy systems, by, for example, facilitating low drift (or drift-free) imaging conditions that enable image acquisition over long time periods.

A first aspect provides a perfusion system for reducing or eliminating sample drift during microscopy imaging. The perfusion system includes a sample chamber that has an inlet opening associated therewith. The perfusion system also includes a reservoir, which is in fluid communication with the inlet opening and positioned above the inlet opening in a direction opposite the force of gravity. In addition, the perfusion system includes an outlet opening associated with the sample chamber. Furthermore, the perfusion system includes a wick. A first portion of the wick forms a fluid-tight connection with the outlet opening. A second portion of the wick is disposed within a waste tank, wherein a capillary tension of the wick contributes to a laminar flow of fluid across an optical detection area of the sample chamber.

The sample chamber of the perfusion system can include various components. For example, in some embodiments, the sample chamber is formed on at least a first side thereof by a coverslip. The coverslip can include a glass coverslip, such as a #1.5 glass coverslip, with, for instance, a diameter between about 10 mm-40 mm. Furthermore, in some embodiments, the inlet opening and the outlet opening are formed within the coverslip, and the outlet opening can be positioned on an opposite side of the coverslip than the inlet opening.

The sample chamber can also be formed on at least a second side thereof by a second coverslip, the second side being opposite the first side of the sample chamber such that the optical detection area is disposed therebetween. The second coverslip can also include a glass coverslip, such as a #1.5 glass coverslip, with, for instance, a diameter between about 10 mm-40 mm. Furthermore, in some embodiments, the second coverslip includes a fiducial coating, such as a Hestzig fiducial coating.

In some embodiments, the perfusion system further includes a spacer positioned between the coverslip and the second coverslip, where the spacer defines a sidewall of the sample chamber. The spacer can be made of or include an inert or nonreactive material, such as a polyimide foil. The spacer can have a thickness between about 5 μm-50 μm (such as a spacer that is less than or equal to about 30 μm in thickness). In combination with the two coverslips, the spacer can define the sample chamber such that it is protected against the ambient gas atmosphere.

The wick of the perfusion system is, in some embodiments, configured to control flow of perfusion media/solution from between about 1 μm/sec 50 μm/sec. The wick can be made of or include a cotton wick. The laminar flow effectuated within the sample chamber may run directionally away from the reservoir and towards the wick, and a substantially even spatial pressure gradient may be formed through a volume of the sample chamber.

A second aspect provides a perfusion system for drift free microscopy imaging. The perfusion system includes a sample chamber defined by a first coverslip, a second coverslip positioned opposite—and in a substantially parallel orientation—to the first coverslip, and a spacer disposed between and abutting the first and second coverslips to form a sidewall of the sample chamber. The perfusion system further includes an inlet opening and an outlet opening formed on opposite sides of the first coverslip, and a reservoir in fluid communication with the inlet opening and positioned above the inlet opening in a direction opposite the force of gravity. The perfusion system also includes a wick associated with a waste tank at a first end and disposed within the outlet opening at a second end. A capillary force of the wick and a pressure difference between the reservoir and the waste tank causes a constant laminar flow of fluid from the inlet opening, through an optical detection area of the sample chamber, and toward the wick while maintaining drift free imaging conditions within the optical detection area over long image acquisition times.

A third aspect provides an imaging system that includes a perfusion system (which may correspond to any perfusion system embodiment or configuration described with reference to the first aspect or the second aspects), one or more optical trains configured to view the optical detection area of the sample chamber, and a sensor for capturing images of the optical detection area of the sample chamber.

In some embodiments, the imaging system further includes a pump or gravity-driven drip reservoir mechanically uncoupled from the perfusion reservoir. The imaging system may be configured for super-resolution imaging. For example, in some instances, the imaging system is adapted for 4-pi 3D localization microscopy.

### BRIEF DESCRIPTION OF THE DRAWINGS

In order to describe the manner in which the above recited and other advantages and features of the disclosure can be obtained, a more particular description of the disclosure briefly described above will be rendered by reference to specific embodiments thereof, which are illustrated in the appended drawings. It is appreciated that these drawings depict only typical embodiments of the disclosure and are not therefore to be considered to be limiting of its scope. The disclosure will be described and explained with additional specificity and detail through the use of the accompanying drawings in which:

FIG. 1A illustrates a top view of an example schematic representation of an exemplary sample chamber configured for use as a component of a perfusion system, in accordance with one or more embodiments of the present disclosure;

FIG. 1B illustrates a side view of the schematic representation of the exemplary sample chamber of FIG. 1A, in accordance with one or more embodiments of the present disclosure;

FIG. 2 illustrates the sample chamber of FIG. 1B as a component of an exemplary perfusion system that includes the sample chamber, a reservoir, and a waste tank, in accordance with one or more embodiments of the present disclosure;

FIG. 3 illustrates an exemplary imaging system that includes the perfusion system of FIG. 2 in association with an optical train and a sensor, in accordance with one or more embodiments of the present disclosure;

FIG. 4 illustrates the sample chamber of FIG. 1B as a component of an exemplary imaging system configured for 4-pi microscopy, in accordance with one or more embodiments of the present disclosure;

FIG. 5A illustrates a top right perspective view of a model of an exemplary housing for a sample chamber configured for use as a component of a perfusion system with the top housing portion thereof aligned over the bottom housing portion; and

FIG. 5B illustrates a bottom left perspective view of the model from FIG. 5A with the top housing portion thereof aligned over the bottom housing portion.

### DETAILED DESCRIPTION

As used in the specification, a word appearing in the singular encompasses its plural counterpart, and a word appearing in the plural encompasses its singular counterpart, unless implicitly or explicitly understood or stated otherwise. Furthermore, it is understood that for any given component or embodiment described herein, any of the possible candidates or alternatives listed for that component may generally be used individually or in combination with one another, unless implicitly or explicitly understood or stated otherwise. Additionally, it will be understood that any list of such candidates or alternatives is merely illustrative, not limiting, unless implicitly or explicitly understood or stated otherwise.

In addition, unless otherwise indicated, numbers expressing quantities, constituents, distances, or other measurements used in the specification and claims are to be understood as being modified by the term “about.” Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

Furthermore, as used in the specification and appended claims, directional terms, such as “top,” “bottom,” “left,” “right,” “up,” “down,” “upper,” “lower,” “proximal,” “adjacent,” “distal,” and the like are used herein solely to indicate relative directions and are not otherwise intended to limit the scope of the specification or claims. In addition, as used in the specification and appended claims, ordinal terms, such as “first,” “second,” “third,” and so forth, are used herein for ease of description and/or for illustrative purposes and are not intended to limit the scope of the specification or claims.

Various embodiments disclosed herein are related to apparatuses, systems, and components associated with perfusion systems for facilitating drift-free microscopy. For example, an exemplary perfusion system disclosed herein includes a sample chamber with inlet and outlet openings associated therewith. The inlet opening is in fluid communication with a reservoir positioned above the inlet opening in a direction opposite the force of gravity. The outlet opening associated with the sample chamber includes a wick that forms a fluid-tight connection with the outlet opening and extends into a waste tank. The capillary tension of the wick and the gravitational force against the fluid in the reservoir contributes to constant laminar flow of perfusion media across an optical detection area of the sample chamber.

Those skilled in the art will recognize, in view of the present disclosure, that the foregoing and at least some other disclosed embodiments address various shortcomings and problems associated with conventional perfusion systems, generally, and provide some distinct advantages over conventional perfusion systems when implemented within drift-free microscopy systems, more specifically. For example, the disclosed perfusion systems utilize capillary forces (e.g., the capillary tension associated with a wick) to facilitate laminar flow of perfusion media through the chamber. This can advantageously avoid the introduction of vibrations to the chamber that are typically introduced by conventional electronic and mechanical perfusion systems. It can also beneficially avoid other problems known in the art—alluded to above—such as heat dissipation, pump and nozzle effects, unintended flow variations, pressure variations, and/or optical deformations of the sample that can negatively impact imaging results when using conventional perfusion systems.

In some embodiments, the relative positioning of the perfusion reservoir, sample chamber, and waste tank achieves a pressure difference via gravity, and this pressure difference helps the capillary force associated with the wick to overcome surface tension and drive laminar flow of media from the perfusion reservoir, through the sample chamber, and into the waste tank in a manner that does not shear or dislodge adherent cells. This beneficially creates a drift-free environment within the sample chamber with continually replenished media—ideal conditions for many optical high-resolution and super-resolution microscopy techniques. Additionally, due to the relative positioning of these components, the pressure difference may reduce over time as the volume of fluid in the waste tank increases and/or the volume of fluid in the reservoir decreases, which may beneficially prevent liquid spills onto associated microscope components.

Accordingly, at least some of the perfusion systems disclosed herein are suitable to facilitate drift-free microscopy and can be particularly useful for 3D localization microscopy, such as 4-pi microscopy. For instance, at least some perfusion systems disclosed herein include a spacer that provides robust and constant spacing between two sample windows and can be implemented with limited space boundary conditions (as may be required for 4-pi imaging). Such perfusion systems can achieve laminar flow characteristics and evenly distribute internal pressures across an optical detection area of the sample chamber. This beneficially enables image collection over long acquisition periods with minimal focus and/or sample drift (e.g., x-y drift) issues. Perfusion systems described herein may also beneficially allow users to uniformly perfuse samples with multiple dyes or probes (e.g., sequentially or simultaneously) for improved multi-color imaging.

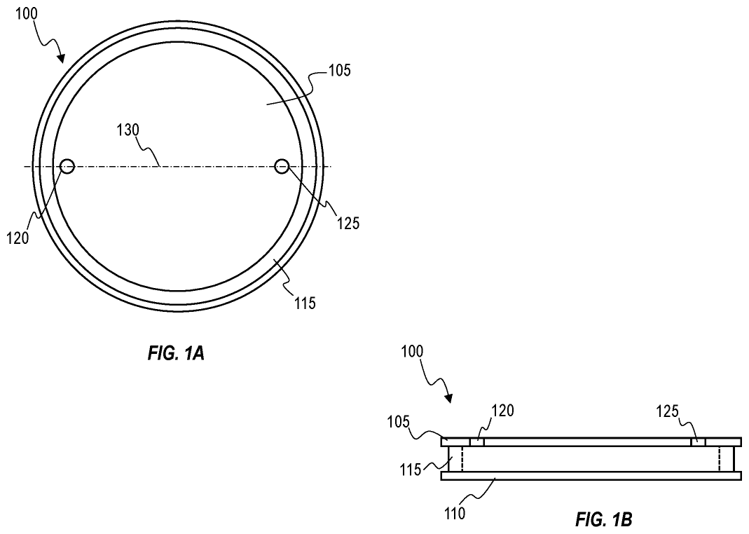

Having just described some of the various high-level features and benefits of the disclosed embodiments, attention will now be directed to FIGS. 1A through 4. These Figures illustrate various conceptual representations, components, systems, and supporting illustrations related to the disclosed embodiments.

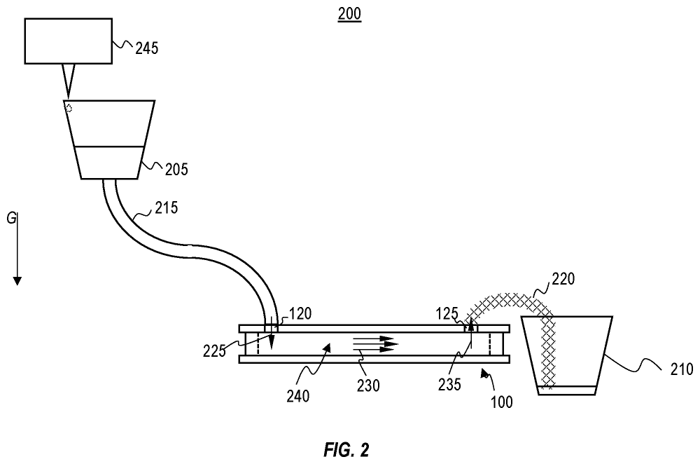

FIGS. 1A and 1B illustrate various views of an example schematic representation of an exemplary sample chamber 100. FIG. 1A provides a top view of the sample chamber 100, and FIG. 1B provides a side view of the sample chamber 100. As described hereinbelow (e.g., with reference to FIG. 2), the sample chamber 100 may be implemented as part of a perfusion system 200 configured to reduce or eliminate sample drift for microscopy imaging.

As indicated above, at least some of the components and/or structures illustrated in FIGS. 1A and 1B (and in other Figures of the present disclosure) are provided, for ease of description, as schematic representations only. Accordingly, the relative scaling and/or specific form of at least some of the components and/or structures as depicted according to the present Figures should not be construed as limiting the principles described herein in any way.

FIGS. 1A and 1B illustrate that, in some embodiments, the sample chamber 100 includes a first coverslip 105 and a second coverslip 110 that are positioned on opposing sides of a spacer 115. Accordingly, the first coverslip 105 may form a top of the sample chamber 100, the second coverslip 110 may form a bottom of the sample chamber 100, and the spacer 115 may form a sidewall of the sample chamber 100.

The first coverslip 105 may be formed of any suitable optically transmissive material, such as glass. For example, in some instances, the first coverslip 105 comprises a #1.5 or #1.5H glass coverslip (e.g., for use with microscope objectives calibrated for imaging samples with a 0.17 mm thick coverslip or culture dish bottom). In other instances, the first coverslip 105 may have a different thickness configured for use with different imaging systems and/or techniques.

FIG. 1A demonstrates that, in some embodiments, the first coverslip 105 is substantially circular in shape. For example, the first coverslip 105 may have a diameter within a range of about 10 mm and 40 mm, such as a diameter of about 35 mm (however, in some instances, the diameter of the first coverslip 105 is smaller than 10 mm or greater than 40 mm). Although FIG. 1A illustrates the first coverslip 105 as having a substantially circular shape, other shapes are within the scope of this disclosure. For example, in some embodiments, the first coverslip 105 is substantially square or rectangular in shape.

As illustrated in FIGS. 1A and 1B, the sample chamber 100 includes an inlet opening 120 and an outlet opening 125, each providing access to the interior portion of the sample chamber 100. FIGS. 1A and 1B illustrate an example embodiment in which both the inlet opening 120 and the outlet opening 125 are formed within the first coverslip 105. In particular, FIGS. 1A and 1B illustrate the inlet opening 120 and the outlet opening 125 formed offset from the center of the first coverslip 105 along the same axis (indicated in FIG. 1A by center line 130), such that the inlet opening 120 and the outlet opening are positioned on substantially opposite sides of the first coverslip 105. By way of explanation, in some instances, the inlet opening 120 and the outlet opening 125 are located on the first coverslip 105 such that a line that passes through the center both the inlet opening 120 and the outlet opening 125 also passes through the center of the first coverslip 105 (e.g., center line 130).

In some instances, the inlet opening 120 and the outlet opening 125 are both offset from the center of the first coverslip 105 by a same distance, whereas, in other instances, the inlet opening 120 and the outlet opening 125 are offset from the center of the first coverslip 105 by different distances. Furthermore, in some instances, the inlet opening 120 and the outlet opening 125 comprise a substantially identical size and/or shape (e.g., circular with a diameter within a range of about 0.1 mm to 5 mm, preferably about 0.5 mm to 2 mm, more preferably about 1 mm), whereas, in other instances, the inlet opening 120 and the outlet opening 125 comprise a different size and/or shape.

As will be described in more detail hereinbelow (e.g., with reference to FIG. 2), the positioning of the inlet opening 120 and the outlet opening 125 proximate to substantially opposing sides of the sample chamber 100 (in combination with other components and/or principles described herein) may facilitate controlled flow, or perfusion, of a liquid medium through the sample chamber 100.

The second coverslip 110 may form a bottom of the sample chamber 100, as more clearly depicted in FIG. 1B. Similar to the first coverslip 105, the second coverslip 110 may be formed of any suitable optically transmissive material, such as glass. As with the first coverslip 105, the second coverslip may comprise a #1.5 or #1.5H glass coverslip, or the second coverslip 110 may have a different thickness configured for use with different imaging systems and/or techniques.

FIGS. 1A and 1B illustrate an embodiment in which the first coverslip 105 and the second coverslip 110 have a substantially similar thickness, shape, and material. For instance, the second coverslip 110 corresponds to the substantially circular shape of the first coverslip 105, and the second coverslip 110 has a diameter that substantially corresponds to that of the first coverslip 105 (e.g., in some instances, within a range of about 10 mm to 40 mm). However, in some embodiments, the second coverslip comprises a different thickness, shape, and/or material as compared to that of the first coverslip 105. For example, in standard microscopy systems (e.g., where imaging occurs from only one side of the sample) for imaging live cells adhered to the second coverslip 110, the second coverslip 110 may comprise a #1.5 or #1.5H glass coverslip, while the first coverslip 105 comprises a different thickness and/or material. In some embodiments, particularly when implemented with 4-pi microscopy systems (e.g., where imaging from opposing sides of the sample), the first coverslip 105 and the second coverslip 110 may comprise substantially identical thicknesses and materials (e.g., both may comprise #1.5 or #1.5H glass coverslips).

Furthermore, in some instances, the second coverslip 110 may include a coating disposed thereon such as a fiducial coating, whereas, in some instances, the first coverslip 105 omits a coating. When used, a fiducial coating may take on various forms, such as a gold nanorod fiducial coating (e.g., provided by Hestzig LLC and/or associated entities) configured to enable the sample chamber 100 for implementation with interferometric photoactivated localization microscopy (iPALM) systems.

As indicated hereinabove, the spacer 115 of the sample chamber 100 forms a sidewall of the sample chamber 100 between the first coverslip 105 and the second coverslip 110. To avoid contamination of the sample chamber 100 and/or specimens situated therein (e.g., live cells), the spacer 115 may comprise an inert or nonreactive material, such as, by way of non-limiting example, a polyimide foil or film material. In some instances, the spacer 115 has a thickness within a range of about 5 μm to about 50 μm, such as a thickness of 8 μm, 16 μm, or another thickness less than 30 μm. The thickness of the spacer 115 can be selected, for example, based on the type of sample to be imaged. For example, a different spacer may be selected when imaging peripheral mononuclear blood cells, which typically have a diameter less than about 10 μm, as compared to imaging some immortal cell culture cell lines that can have an average diameter greater than about 30 μm (e.g., HeLa cells). In some instances, the thickness of the spacer 115 is smaller than 5 μm or greater than 50 μm. The spacer 115 may be configured to provide an even thickness or volume throughout the sample chamber, which may allow the first coverslip 105 and the second coverslip 110 to be arranged in abutment with the spacer 115 in a substantially parallel manner.

With the first coverslip 105 and the second coverslip 110 arranged in abutment with the spacer 115 on opposing sides thereof, an optical detection area or region may become formed/disposed between the first coverslip 105 and the second coverslip 110 and between the inlet opening 120 and the outlet opening 125.

As indicated above, the sample chamber 100 described with reference to FIGS. 1A and 1B can be implemented as part of a perfusion system 200 (see, briefly, FIG. 2) configured to reduce or eliminate sample drift for microscopy imaging. A perfusion system 200 may operate based on pressure differences between different components of the perfusion system in combination with capillary force of a wick to facilitate controlled, drift-free flow of liquid media through the optical detection area of the sample chamber 100. In some instances, the selection and uniformity of the thickness of the spacer 115 (e.g., within a range of about 5 μm to about 50 μm) and the parallel arrangement of the first coverslip 105 and the second coverslip 110 (e.g., according to the example embodiment shown and described with reference to FIGS. 1A and 1B) may facilitate a substantially even spatial pressure gradient and/or an even flow of a liquid medium through the optical detection area of the sample chamber 100 during perfusion operations (e.g., utilizing other components of the perfusion system 200).

However, as indicated above, aspects of the sample chamber 100 may be modified to provide different flow characteristics of liquid media through the sample chamber 100 during perfusion operations (e.g., utilizing other components of the perfusion system 200). For example, the shape of the first coverslip 105, the second coverslip 110, and/or the spacer 115 may be modified (e.g., resulting in substantially square or rectangular sample chambers 100) to provide modified flow characteristics through the optical detection area of the sample chamber 100 and thereby enable sample differentiation within the experimental volume of the sample chamber 100. Furthermore, the thickness of the spacer 115 may be modified, thereby modifying the aspect ratio of the profile of the sample chamber 100 and providing modified flow characteristics through the optical detection area of the sample chamber 100, enabling sample differentiation within the experimental volume of the sample chamber 100. Still furthermore, the positioning of the inlet opening 120 and/or the outlet opening 125 may be modified (e.g., distance from the center of the sample chamber 100, arrangement along the same center line 130, etc.) to facilitate modified flow characteristics through the optical detection area of the sample chamber 100, thereby enabling sample differentiation within the experimental volume of the sample chamber 100.

Additional details concerning the operation of the perfusion system 200 will now be provided with reference to FIG. 2, which illustrates an example schematic representation of components of a perfusion system 200. In particular, FIG. 2 illustrates that a perfusion system 200 includes a sample chamber 100 (e.g., as described hereinabove with reference to FIGS. 1A and 1B), a reservoir 205, and a waste tank 210. The reservoir 205 is in fluid communication with the inlet opening 120, which is depicted in FIG. 2 by the conduit 215 extending from the reservoir 205 to the inlet opening 120. FIG. 2 furthermore shows that the reservoir is positioned above the inlet opening 120 and the waste tank 210 in a direction opposite the force of gravity (indicated in FIG. 2 by the arrow G). The force of gravity, therefore, creates a pressure difference between the reservoir 205 and the other portions of the perfusion system 200 (e.g., the sample chamber 100 and the waste tank 210).

For illustrative purposes, FIG. 2 depicts a liquid medium within the reservoir 205 and flowing through the sample chamber 100 into the waste tank 210. The flow of the liquid medium from the reservoir 205 and through the sample chamber 100 into the waste tank 210 may be facilitated through the relative positioning of the components of the perfusion system 200 as well as the capillary tension of a wick 220 of the perfusion system 200.

FIG. 2 illustrates the wick 220 of the perfusion system 200 extending between the sample chamber 100 and the waste tank 210. In particular, one portion of the wick 220 forms a fluid-tight connection with the outlet opening 125 of the sample chamber 100, and another portion of the wick 220 is disposed within the waste tank 210. In some instances, the structure of the wick 220 enables the wick 220 to cause a capillary effect, whereby intermolecular forces between the molecules of the liquid medium and the surfaces/fibers of the wick 220 overcome the surface tension of the liquid medium (i.e., the cohesive forces between the molecules of the liquid medium), causing the liquid medium to adhere to the wick 220 and be drawn therethrough.

Furthermore, the relative positioning of the components of the perfusion system 200 (e.g., the reservoir 205, the sample chamber 100, the waste tank 210) create, via gravity, a regular pressure difference within the sample chamber 100. For example, gravity may cause a substantially even spatial pressure gradient between the inlet opening 120 (connected to the elevated reservoir 205, providing higher pressure) and the outlet opening 125 (connected to the lower waste tank via the wick 220, providing lower pressure) of the sample chamber 100. Accordingly, gravity may support a weak but consistent pressure difference within the sample chamber 100, which may also contribute to the capillary forces of the wick 220 overcoming the surface tension of the liquid medium.

Thus, the capillary tension of the wick 220 (and, in at least some instances, the pressure difference brought about by the relative elevation of the reservoir 205 and the waste tank 210) may cause laminar flow (i.e., non-turbulent, symmetric flow in the z-direction of the sample chamber 100) of the liquid medium from the inlet opening 120 (indicated by flow arrow 225) through an optical detection area 240 of the sample chamber 100 (indicated by flow arrows 230) toward the wick 220, where the wick 220 draws the liquid medium into the waste tank 210 (indicated by flow arrow 235).

Accordingly, in some instances, a perfusion system 200 may achieve laminar flow with even or controlled flow characteristics (e.g., minimal flow fluctuations) throughout the optical detection area 240 from the inlet opening 120 (or from the reservoir 205) toward the outlet opening 125 (or toward the wick 220) of the sample chamber 100. In this regard, the perfusion system 200 may provide drift-free imaging conditions within the optical detection area 240, which may be persisted over long image acquisition times for implementation with standard or 4-pi microscopy experimentation (e.g., within the minute range or up to or exceeding one hour). In one example, users may employ a perfusion system 200 as disclosed herein to perfuse fixed and live adherent cells (e.g., within the optical detection area 240 of the sample chamber 100) with a buffer (or another liquid medium) for optical high-resolution or super-resolution microscopy (or for other purposes).

In some instances, the wick 220 is configured to control flow at a rate within a range of about 1 μm/sec to about 50 μm/sec. The wick may comprise any suitable material, such as, by way of non-limiting example, cotton. Furthermore, it should be noted that, as indicated above, the reservoir 205 and the waste tank 210 (as well as other portions of FIGS. 2-4) are illustrated as conceptual representations thereof, and the reservoir 205 and the waste tank 210 may take on any suitable form. For example, although FIG. 2 illustrates the reservoir 205 and the waste tank 210 as having a substantially similar volume (e.g., 10 ml or another value), the reservoir 205 and the waste tank 210 may have different volumes than one another.

FIG. 2 also illustrates that, in some instances, the perfusion system 200 may be associated with an external reservoir 245, which may be operable for manual or automatic refilling of the reservoir 205 as desired. The external reservoir 245 may be implemented, in some instances, as a pump or gravity-driven drip reservoir. Importantly the external reservoir 245 is mechanically uncoupled from the other components of the perfusion system 200, which may prevent vibrations caused by operation of the external reservoir 245 from affecting the flow characteristics through the liquid medium through the sample chamber 100 (in particular the optical detection area 240 thereof). Furthermore, providing an external reservoir 245 that is mechanically uncoupled from the other components of the perfusion system 200 may prevent heat dissipation from the external reservoir 245 to the sample chamber 100, thereby preventing the external reservoir 245 from altering the thermal characteristics of the sample and/or influencing the biochemical reactivity exhibited by a sample within the sample chamber 100.

Furthermore, by avoiding apparatuses such as pumps, pipettes, syringes, and the like, the perfusion system 200 of the present disclosure provides a technique for perfusing a sample chamber 100 in a manner that avoids excessive external wiring, tubing, and/or instruments that may, during operation, apply mechanical stresses to the sample chamber, which may cause drift. The perfusion system 200 may also avoid vibrations, nozzle effects, and/or other effects that may cause sample drift during perfusion operations.

In addition, the perfusion system 200 may protect the sample chamber 100 thereof from external gases that may affect microscopy experiments (e.g., buffers may be affected by oxygen levels). For example, an oil layer or other sealing agent may be disposed over the liquid medium within the reservoir 205 to prevent contamination of the liquid medium prior to entry into the sample chamber 100.

In some embodiments, as illustrated in FIG. 2, the waste tank 210 is arranged such that when the waste tank 210 becomes full, the liquid level within the waste tank becomes balanced with the liquid level within the sample chamber 100, thereby reducing or eliminating the pressure difference between the outlet opening 125 and the waste tank 210. Accordingly, flow of the liquid medium through the sample chamber 100 may significantly slow or stop as the liquid level within the waste tank 210 approaches the liquid level of the sample chamber 100. Such an arrangement may provide additional benefits, such as preventing spills of liquid media onto microscope optics or other portions of a microscope system or experimental setup. However, it should be noted that other relative positionings of the waste tank 210 and the sample chamber 100 are within the scope of this disclosure. For example, the waste tank 210 may be positioned such that the top of the waste tank 210 is below the liquid level of the sample chamber 100, such that flow will continue into the waste tank 210 via the wick 220 even if the waste tank 210 were to become full.

Furthermore, those skilled in the art will recognize, in view of the present disclosure, that the relative positioning of the different components of the perfusion system 200 (e.g., the reservoir 205, the sample chamber 100, the waste tank 210) may be altered to provide modified pressure differences throughout different portions of the perfusion system, thereby facilitating different flow characteristics (e.g., flow speed) of liquid medium through the optical detection area 240 of the sample chamber 100. The use of different wicks 220 with different capillary tensions may also affect the rate of fluid flow through the sample chamber 100. Also, as noted above, aspects of the sample chamber 100 may also be modified to provide different flow characteristics through the optical detection area 240 of the sample chamber 100 (e.g., size, shape, and/or material of the first coverslip 105, the second coverslip 110, or the spacer 115, or the positioning and/or size of the inlet opening 120 or the outlet opening 125). Additional or alternative variations are also within the scope of the present disclosure, such as positioning the outlet opening 125 on the second coverslip 110 to facilitate more rapid fluid flow through the perfusion system 200.

It should be noted that a perfusion system 200 may include other components not explicitly illustrated in FIGS. 1A, 1B, and 2. For example, a sample chamber 100 of a perfusion system 200 may further comprise a lower casing, lower seal, upper casing, and upper seal. The lower seal may be positioned below and in abutment with the second coverslip 110, while the lower casing is positioned below and in abutment with the lower seal. Similarly, the upper seal may be positioned above and in abutment with the first coverslip 105, while the upper casing is positioned above and in abutment with the upper seal. The upper casing and the lower casing may be fastened together with screws (e.g., M2 screws) to press the upper seal against the first coverslip 105 and to press the lower seal against the second coverslip 110, thereby pressing the first coverslip 105 and the second coverslip 110 against the spacer 115 positioned therebetween, forming the volume of the sample chamber 100. The lower casing and the upper casing may be formed machined of any suitable, rigid material, such as aluminum, titanium, and/or others. Also, the lower seal and the upper seal may be formed of any suitable material, such as a material suitable for exerting force on glass (e.g., silicone, rubber, and/or others).

As noted hereinabove, a perfusion system 200 may be implemented with various microscopy systems to facilitate a low-drift or drift-free environment for high-resolution live cell imaging, such as photoactivated localization microscopy (e.g., multicolor fluorescent imaging) The perfusion system 200 may be implemented with imaging systems that employ various techniques, such as, by way of non-limiting example, super-resolution techniques (e.g., confocal imaging, 4-pi imaging), TIRF (total internal reflection fluorescence), and/or others. For example, the perfusion system 200 may facilitate drift-free, laminar flow of a liquid medium through the optical detection area 240 thereof for significant time periods (e.g., supporting experiments up to an hour or longer in some embodiments), thereby enabling implementation with high-resolution laser scanning methods (e.g., enabling the acquisition of 10,000 to 100,000 frames) or other methods (e.g., structured illumination imaging) for 3D localization microscopy.

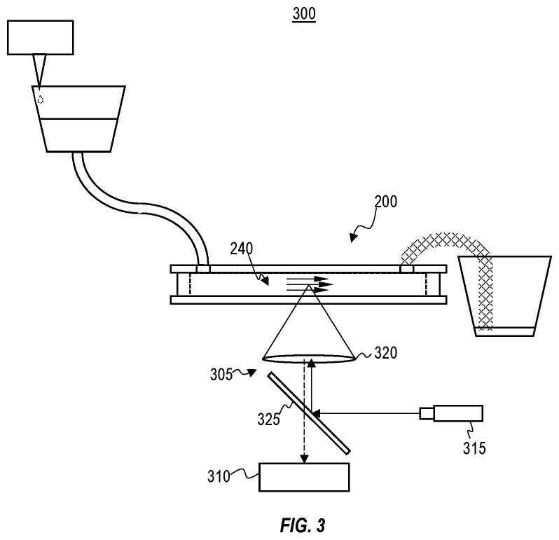

FIG. 3 illustrates an example schematic representation of an imaging system 300 that includes a perfusion system 200, an optical train 305, and an image sensor 310. Reference labeling is omitted from at least some portions of the perfusion system 200 for clarity.

The optical train 305 may comprise one or more optical elements configured to facilitate viewing of the optical detection area 240 of the perfusion system 200 by directing light from a light source 315 toward the optical detection area 240 to illuminate the optical detection area 240. The optical train 305 may also be configured to direct light scattered, reflected, and/or emitted by a specimen within the optical detection area 240 toward the image sensor 310. The light source 315 may be configured to emit various types of light, such as white light or light of one or more particular wavelength bands. In some embodiments (e.g., where the specimen within the optical detection area 240 includes fluorophores), the light source 315 may comprise a fluorophore excitation light source. For example, the light source 315 may comprise a light engine comprising multiple light emitting diodes (LEDs) configured to emit one or more excitation wavelengths (excitation light) for causing fluorophores within the optical detection area 240 of the perfusion system 200 to emit light (emission light).

As a general method of operation, a fluorophore excitation source can be automatically or manually directed to provide multiple bandwidths of light ranging from violet (e.g., 380 nm) to near infrared (e.g., at least 700 nm) and are designed to excite fluorophores, such as, for example, cyan fluorescent protein (CFP) and Far Red (i.e., near-IR) fluorophores. Example LED bandwidths with appropriate excitation filters (e.g., as selected via a computer driven excitation filter wheel) can include, but are not limited to, Violet (380-410 nm LED & 386/23 nm excitation filter), Blue (420-455 nm LED & 438/24 nm excitation filter), Cyan (460-490 nm LED & 485/20 nm excitation filter), Green (535-600 nm LED & 549/15 nm excitation filter), Green (535-600 nm LED & 560/25 nm excitation filter), Red (620-750 nm LED & 650/13 nm excitation filter), and Near-IR (700-IR nm LED & 740/13 nm excitation filter). The two Green/excitation filter combinations listed above can be provided optionally via, for example, a mechanical flipper, when desiring to improve the brightness of red and scarlet dyes. Of course, other LED bandwidths can also be used.

The optical train 305 may comprise one or more optical filters 325 (e.g., which may be implemented as a single filter or in other forms, such as a multi-position dichroic filter wheel and/or a multi-position emission filter wheel) that selectively reflect the excitation light from the light source 315 (e.g., represented in FIG. 3 by solid arrows reflecting off of the optical filter(s) 325) while selectively transmitting the emission light from fluorophores within the optical detection area 240 (e.g., represented in FIG. 3 by dashed arrows transmitting through the optical filter(s) 325), or vice-versa. The optical train may also comprise one or more objective lenses 320 to focus the excitation light from the light source 315 within the optical detection area 240. In some instances, the objective lens(es) 320 comprise a high numerical aperture objective configured for super-resolution microscopy.

As noted, the optical train 305 may be configured to direct light scattered, reflected, and/or emitted from the optical detection area 240 of the perfusion system 200 toward an image sensor 310. The image sensor 310 may be configured to capture image data representative of the specimen positioned within the optical detection area 240. The image data captured by the image sensor 310 may be further analyzed to create a three-dimensional (3D) representation of the specimen positioned within the optical detection area 240. In some instances, the image sensor 310 is in communication with a computing system to facilitate processing of the image data (e.g., to construct the 3D representation of the specimen). The computing system can additionally be used as a controller for the system as well as for performing an analysis and/or storage of data obtained by image sensor 310. Computing systems can comprise a general purpose or specialized computer, server, or any other computing center (e.g., whether local or remote).

In some instances, the imaging system 300 may be configured to adjust the relative positioning of the optical detection area 240 of the perfusion system 200 and an image sensor assembly (e.g., comprising the light source 315, the optical train 305, and the image sensor 310) to scan the optical detection area 240 from different positions (e.g., different x, y, and/or z positions, wherein the x-y plane is parallel to the surface of the image sensor 310 and the z-direction is orthogonal to the surface of the image sensor) to capture image data for constructing a 3D representation of the contents of the optical detection area 240 (e.g., including the positions of fluorophores within the optical detection area 240).

In this regard, the imaging system 300 may include other components not explicitly illustrated in FIG. 3. For example, an imaging system may include a stage assembly and/or positioning mechanism configured to retain and selectively adjust the relative positioning (e.g., in the x, y, and/or z dimension) of the optical detection area 240 of the perfusion system 200 and the image sensor assembly to position different portions of the optical detection area 240 within the field of view of the image sensor 310. The stage assembly and/or positioning mechanism may operate using any techniques known in the art, such as, for example, a stepper motor and screw/nut combination providing step-wise movements of the sample in increments (e.g., down to 0.006 μm/microstep).

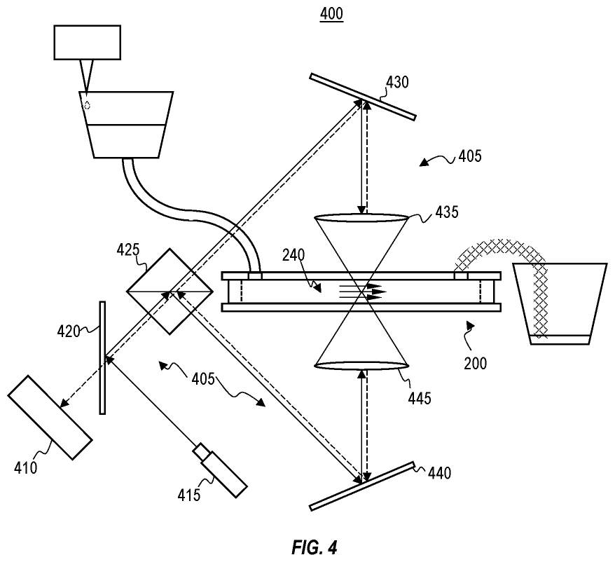

FIG. 4 illustrates an example schematic representation of an imaging system 400 configured for 4-pi microscopy (e.g., interferometric, confocal, etc.). In a general sense, the imaging system 400 may be similar, in at least some respects, to the imaging system 300 described hereinabove with reference to FIG. 3. In particular, the imaging system 400 also includes a perfusion system 200, one or more optical trains 405, and an image sensor 410. Advantageously, the perfusion system 200 as described hereinabove may facilitate controlled flow of fluid through the sample chamber 100 in a manner that omits pumps, syringes, pipettes, and/or other flow-inducing instruments from the top portion of the sample chamber 100 immediately above the optical detection area 240. Accordingly, the perfusion system 200 may provide an un-occluded view of the optical detection area 240 from both sides thereof, enabling the perfusion system to be implemented with 4-pi microscopy systems.

The one or more optical trains 405 comprise one or more optical elements configured to facilitate viewing of the optical detection area 240 of the sample chamber 100 from multiple sides. In particular, the one or more optical trains 405 are configured to direct light from a light source 415 (e.g., a fluorophore excitation light source) toward opposing sides of the optical detection area 240 of the sample chamber 100 simultaneously. For example, fluorophore excitation light emitted from the light source 415 (represented in FIG. 4 by solid arrows originating from the light source 415) may be selectively reflected off of one or more optical filters 420 and split via a beam splitter 425. One portion of the split fluorophore excitation light may then reflect off of a mirror 430 toward an objective lens 435 on one side of the optical detection area 240, while another portion of the split fluorophore excitation light may reflect off of a mirror 440 toward an objective lens 445 on an opposing side of the optical detection area 240. Fluorophore emission light emitted by fluorophores within the optical detection area 240 (represented in FIG. 4 by dashed arrows) may then propagate from the optical detection area 240 and be directed by the objective lenses 435, 445, mirrors 430, 440, beam splitter 425, and one or more optical filters 420 toward the image sensor 410. The image sensor 410 may collect image data from the received light. Accordingly, an imaging system 400 may perform scanning operations to collect image data representative of different portions of the optical detection area 240 to construct a super-resolution 3D representation of a specimen within the optical detection area 240.

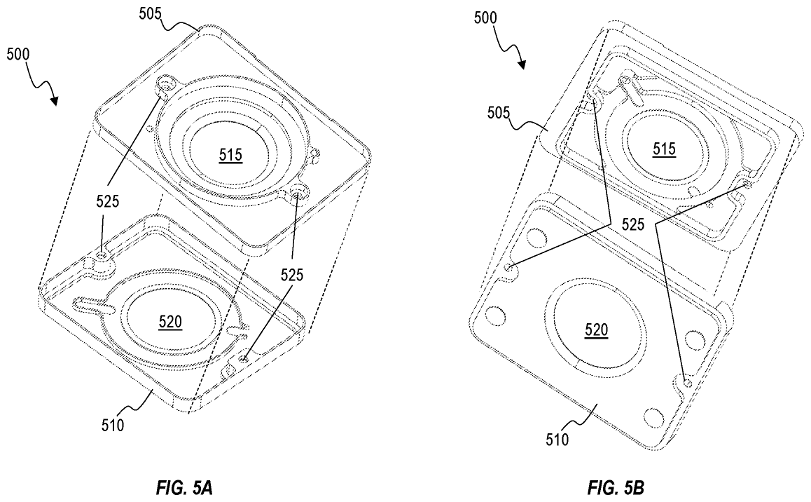

Referring now to FIGS. 5A and 5B, illustrated are perspective views of a model of an exemplary housing 500 for a sample chamber configured for use as a component of the perfusion systems disclosed herein. As shown in each of FIGS. 5A and 5B, the top housing portion 505 is aligned over the bottom housing portion 510. A sample chamber akin to that shown in FIGS. 1A and 1B can be assembled using the housing 500 illustrated in FIGS. 5A and 5B by placing a top coverslip (not shown) over the top aperture 515 of the top portion 505 of the housing 500, placing a bottom coverslip (not shown) over the bottom aperture 520 of the bottom portion 510 of the housing 500, and securing a spacer (not shown) between the coverslips by connecting the top portion 505 and bottom portion 510 of the housing 500. The housing portions can be connected using any means known in the art. As shown in FIGS. 5A and 5B, the housing portions can be connected by passing a bolt or other threaded attachment member through corresponding threaded bores 525 in the respective portions of the housing 500.

It should be appreciated that housing configurations other than that shown in FIGS. 5A and 5B are included within the scope of this disclosure and that the housing 500 is exemplary in nature. For example, the housing portions can be connected using other attachment mechanisms known in the art, such as opposing biasing members, adhesives, magnets, rivets, and the like. The housing can be reusable or disposable and can be made of any suitable material known in the art, from metals to thermoplastics. Additionally, in some embodiments, the apertures can be a different shape than the circular apertures shown in FIGS. 5A and 5B. For example, the aperture can be defined by sides that are polygonal, arcuate, or a combination of the two, according to the desired shape of the viewing window into the sample chamber.

It is to be understood that features described with regard to the various embodiments herein may be mixed and matched in any desired combination. In addition, the concepts disclosed or envisioned herein may be embodied in other specific forms. The described embodiments are to be considered in all respects only as illustrative and not restrictive. The scope is, therefore, indicated by the appended claims rather than by the foregoing description. All changes which come within the meaning and range of equivalency of the claims are to be embraced within their scope.

1. A perfusion system for reducing or eliminating sample drift during microscopy imaging, comprising:
a sample chamber having an inlet opening associated therewith;
a reservoir in fluid communication with the inlet opening and positioned above the inlet opening in a direction opposite the force of gravity;
an outlet opening associated with the sample chamber; and
a wick having a first portion thereof forming a fluid tight connection with the outlet opening and having a second portion thereof disposed within a waste tank, wherein a capillary tension of the wick contributes to a laminar flow of fluid across an optical detection area of the sample chamber.

2. The perfusion system of claim 1, wherein the sample chamber is formed on at least a first side thereof by a glass coverslip, and wherein the coverslip has a diameter between about 10 mm-40 mm.

3-5. (canceled)

6. The perfusion system of claim 2, wherein the inlet opening and the outlet opening are formed within the coverslip.

7. The perfusion system of claim 6, wherein the outlet opening is positioned on an opposite side of the coverslip than the inlet opening.

8. The perfusion system of claim 2, wherein the sample chamber is formed on at least a second side thereof by a second glass coverslip, the second side being opposite the first side of the sample chamber such that the optical detection area is disposed therebetween.

9. (canceled)

10. The perfusion system of claim 8, wherein the second coverslip has a diameter between about 10 mm-40 mm.

11. (canceled)

12. The perfusion system of claim 8, wherein the second coverslip includes a fiducial coating.

13. The perfusion system of claim 8, further comprising a spacer positioned between the coverslip and the second coverslip, the spacer defining a sidewall of the sample chamber.

14. The perfusion system of claim 13, wherein the spacer is made of or includes an inert or nonreactive material.

15. The perfusion system of claim 13, wherein the spacer comprises a polyimide foil having a thickness between about 5 μm-50 μm.

16. The perfusion system of claim 13, wherein the thickness of the spacer is less than or equal to about 30 μm.

17. The perfusion system of claim 1, wherein the sample chamber is protected against ambient gas atmosphere.

18. The perfusion system of claim 1, wherein the wick is configured to control flow from between about 1 μm/sec-50 μm/sec.

19. The perfusion system of claim 1, wherein the wick comprises a cotton wick.

20. The perfusion system of claim 1, wherein the laminar flow runs directionally away from the reservoir and towards the wick.

21. The perfusion system of claim 1, wherein a substantially even spatial pressure gradient is formed through a volume of the sample chamber.

22. A perfusion system for drift free microscopy imaging, comprising:
a sample chamber defined by a first coverslip, a second coverslip positioned opposite—and in a substantially parallel orientation—to the first coverslip, and a spacer disposed between and abutting the first and second coverslips to form a sidewall of the sample chamber;
an inlet opening and an outlet opening formed on opposite sides of the first coverslip;
a reservoir in fluid communication with the inlet opening and positioned above the inlet opening in a direction opposite the force of gravity; and
a wick associated with a waste tank at a first end and disposed within the outlet opening at a second end,
wherein a capillary force of the wick and a pressure difference between the reservoir and the waste tank causes a laminar flow of fluid from the inlet opening, through an optical detection area of the sample chamber, and toward the wick while maintaining drift free imaging conditions over long image acquisition times.

23. An imaging system, comprising:
a perfusion system comprising:
a sample chamber having an inlet opening associated therewith;
a reservoir in fluid communication with the inlet opening and positioned above the inlet opening in a direction opposite the force of gravity;
an outlet opening associated with the sample chamber; and
a wick having a first portion thereof forming a fluid tight connection with the outlet opening and having a second portion thereof disposed within a waste tank, wherein a capillary tension of the wick contributes to a laminar flow of fluid across an optical detection area of the sample chamber;
one or more optical trains configured to view the optical detection area of the sample chamber; and
a sensor for capturing images of the optical detection area of the sample chamber, and a pump or gravity driven drip reservoir mechanically uncoupled from the perfusion system.

24. The imaging system of claim 23, wherein the imaging system is configured for super-resolution imaging.

25. The imaging system of claim 23, wherein the imaging system is adapted for 4-pi 3D localization microscopy.

26. (canceled)
